(19) United States
(12) Patent Application Publication
(10) Pub. No.: US 2025/0136947 A1
(43) Pub. Date: May 1, 2025

(54) IN VITRO CO-CULTURE ALVEOLUS-CAPILLARY MODEL SYSTEM FOR STUDYING ACUTE LUNG INJURY INDUCED BY RESPIRATORY VIRUS INFECTIONS

(71) Applicant: Centre for Immunology & Infection Limited, Hong Kong (HK)

(72) Inventors: Chi Wai Michael CHAN, Hong Kong (HK); Po Yee MA, Hong Kong (HK); Hiu Ha CHING, Hong Kong (HK)

(21) Appl. No.: 18/922,390

(22) Filed: Oct. 22, 2024

Related U.S. Application Data
(60) Provisional application No. 63/593,243, filed on Oct. 26, 2023.

(51) Int. Cl. C12N 5/071; A61K 35/35; C12Q 1/70; G01N 33/50

(52) U.S. Cl. CPC C12N 5/0688; A61K 35/35; C12Q 1/70; G01N 33/5044; C12N 2502/28; C12N 2503/04; C12N 2509/00; C12N 2533/54; G01N 2500/10

(57) ABSTRACT

The present invention provides a co-culture system of human alveolar epithelial and endothelial cells to study the interactions within the epithelial-endothelial barrier in the lungs. It demonstrates that infection with highly pathogenic influenza A virus A/HK/483/97 (H5N1) significantly decreases alveolar fluid clearance (AFC) and increases protein permeability (APP). The administration of therapeutic extracellular vesicles (EVs) after infection effectively restores AFC and APP, reduces viral replication, and enhances the expression of anti-viral genes. This invention highlights the therapeutic potential of EVs and offers a valuable tool for investigating acute lung injury and improving lung drug delivery techniques.

### CROSS-REFERENCE TO RELATED APPLICATIONS

The present application claims the priorities from the U.S. provisional patent application Ser. No. 63/593,243 filed Oct. 26, 2023, and the disclosure of which is incorporated herein by reference in its entirety.

### FIELD OF THE INVENTION

This invention utilizes primary cells isolated from human lung tissues seeded into a transwell system, which increases the physiological relevance of the in vitro lung model and allows for the investigation of cellular interactions in the alveolus-capillary barrier outside of the clinical setting.

### BACKGROUND OF THE INVENTION

In severe cases of respiratory diseases, vascular leakage and lung oedema contributes to rapid disease progressing, ultimately leading to respiratory failure. Research indicates that damage to the respiratory endothelium leads to cell infiltration and fluid leak into the alveolar spaces. On the other hand, a healthy respiratory epithelium provides a physical and immunological barrier against exterior pathogens, which protects the underlying tissues from direct infection. However, in diseased states, the integrity of the epithelium is compromised, resulting in damage to the endothelium and allowing pathogens to enter the bloodstream. Therapeutic strategies to address these issues include restoring epithelial and endothelial integrity through tight/adherens junctions and modifying cytoskeletal arrangements via specific signaling pathways.

With the advent of the organ-on-a-chip (OOAC) culture system, mechanical stretching can be applied to the AA battery-sized system to better mimic the physiological movement of lung tissues during breathing. However, it has been reported that responses of a lung-on-a-chip still cannot fully reproduce the responses observed in native alveolar epithelial cells (AECs). In terms of drug testing, since the dimension of the microfluidic system is very small, the surface effect can dominate the volume effect, which can cause more drug particles to be adsorbed than expected, disrupting the mixture of fluids at intersections. Also, special equipment may be required to increase data reliability when OOAC is used in modelling the cancer environment or the human intestine.

A predictive in vitro modelling system that accurately resembles human physiological conditions is essential for understanding the mechanisms driving disease pathogenesis outside clinical settings. Currently in vitro models often consist of monocultures, which may behave distinct from those observed in vivo due to the absence of microenvironmental interactions.

Moreover, the ethical concerns surrounding animal models and their generally low predictive value for human diseases underscore the need for alternative approaches. While immortalized cell lines, such as A549 cells derived from human lung carcinoma, have been widely used, their phenotypic differences arising from in vitro culture conditions limit their ability to replicate critical features of the physiological epithelium.

Investigating the physiological changes and related mechanisms of respiratory virus-induced acute lung injury outside clinical settings is challenging. Accurately modeling these processes in vitro with physiological relevance is crucial for advancing drug screening efforts, particularly for precision medicine. Developing a more representative model could facilitate the exploration of therapeutic interventions and enable better predictions of clinical outcomes, ultimately leading to improved patient care.

### SUMMARY OF THE INVENTION

The present invention aims to enhance the physiological relevance of the lung model, and therefore a co-culture model has been established. This invention discloses a co-culture in vitro lung injury format optimized for primary human AECs and human lung microvascular endothelial cells (HMVECs) cultures for investigating mechanisms of highly pathogenic influenza virus infection pathogenesis and recovery.

The human AECs and HMVECs are cultured on collagen in a transwell system. This lung injury model can be used to study the changes in alveolar fluid clearance (AFC) and alveolar protein permeability (APP) upon respiratory virus infection, both of which are important contributors to the fatal development of acute respiratory distress syndrome (ARDS) arising from severe respiratory virus infections.

In a first aspect, the present invention provides an in vitro co-culture alveolus-capillary model system for studying acute lung injury induced by respiratory virus infections. The system includes a transwell system with primary human alveolar epithelial cells seeded onto an apical chamber surface of the transwell system and human lung microvascular endothelial cells seeded onto a basolateral surface of the transwell system, and an apparatus for measuring alveolar fluid clearance and alveolar protein permeability in infected cells. The in vitro co-culture alveolus-capillary model system mimics an alveolo-capillary barrier.

In one embodiment, the primary human alveolar epithelial cells are cultured on a collagen-coated transwell insert to promote tight junction formation.

In one embodiment, the primary human alveolar epithelial cells are isolated from a non-malignant lung tissue.

In one embodiment, the primary human alveolar epithelial cells are seeded at a cell density of 5×10⁴ to 2×10⁵ cells per well, and the human lung microvascular endothelial cells are seeded at a cell density of 5×10⁴ to 2×10⁵ cells per well.

In one embodiment, the transwell system has a pore size of 0.4 μm.

In a second aspect, the present invention provides a method for in vitro studying acute lung injury induced by respiratory virus infections, including: isolating primary human alveolar epithelial cells and human microvascular endothelial cells; seeding the primary human alveolar epithelial cells on an apical chamber surface of a transwell membrane and seeding the human microvascular endothelial cells on a basolateral surface of the transwell system to form an in vitro co-culture alveolus-capillary model system; infecting the in vitro co-culture alveolus-capillary model system with one or more respiratory virus; and assessing effect of viral infection on at least one physiological parameter comprising AFC and APP.

In one embodiment, the primary human alveolar epithelial cells and the human microvascular endothelial cells are isolated using enzymatic digestion followed by density gradient centrifugation and magnetic bead separation.

In one embodiment, the primary human alveolar epithelial cells are cultured on a collagen-coated transwell insert to promote tight junction formation.

In one embodiment, the primary human alveolar epithelial cells are isolated from a non-malignant lung tissue.

In one embodiment, the primary human alveolar epithelial cells are seeded at a cell density of 5×10⁴ to 2×10⁵ cells per well, and the human lung microvascular endothelial cells are seeded at a cell density of 5×10⁴ to 2×10⁵ cells per well.

In one embodiment, the transwell system has a pore size of 0.4 μm.

In one embodiment, the one or more respiratory virus are selected from the group consisting of coronaviruses, respiratory syncytial viruses, adenoviruses, influenza viruses, parainfluenza viruses, rhinoviruses, enteroviruses, and herpes simplex viruses.

In one embodiment, the in vitro co-culture alveolus-capillary model system is infected by influenza A virus subtype H5N1.

In another embodiment, the method further includes adding a therapeutic agent after the viral infection to inhibit viral replication within both the human alveolar epithelial cells and the human lung microvascular endothelial cells.

In one embodiment, the therapeutic agent includes extracellular vesicles (EVs) in a concentration of 1×10⁷ to 1×10⁹ EV particles per well. The extracellular vesicles are derived from human mesenchymal stromal cells.

In another embodiment, the method further includes measuring transepithelial electrical resistance (TEER) as an indicator to assess the integrity of the epithelial-endothelial barrier over time, and the TEER is maintained at ≥800 Ω/cm².

In one embodiment, the step of assessing effect of viral infection on at least one physiological parameter further includes measuring viral gene expression and anti-viral gene expression in both the primary human alveolar epithelial cells and the human microvascular endothelial cells.

### BRIEF DESCRIPTION OF THE DRAWINGS

Embodiments of the invention are described in more details hereinafter with reference to the drawings, in which:

FIG. 1A shows a schematic representation of the in vitro co-culture alveolus-capillary model system using confluent monolayers of human AECs seeded onto the collagen-coated apical chamber and human HMVECs seeded onto the underside of the membrane of each transwell to mimic the human alveolus and surrounding capillaries in clinical setting;

FIG. 1B demonstrates that the integrity of tight junctions in the cells is assessed daily by measuring transepithelial electrical resistance with an epithelial voltohmmeter (EVOM) machine;

FIG. 1C depicts TEER values of the alveolo-capillary barrier observed across a six-day period before experiment;

FIG. 1D shows infection of AECs with the highly pathogenic influenza virus A (H5N1) results in a significant reduction in AFC and an increase in APP. Data represent the mean±SD from three experiments. * p≤0.05; ** p≤0.01 and *** p≤0.001;

FIG. 2A shows that EV treatment of the infected cells restores AFC across the barriers compared to untreated infected cells. Data represent the mean±SD from three experiments. * p≤0.05; ** p≤0.01 and *** p≤0.001;

FIG. 2B shows that EV treatment of the infected cells decreases APP across the barriers compared to untreated infected cells. Data represent the mean +SD from three experiments. * p≤0.05; ** p≤0.01 and *** p≤0.001;

FIG. 2C shows A (H5N1)-infected AECs and A (H5N1)-infected HMVECs display high expression of influenza viral M-gene. EV treatment suppresses viral replication across the barrier. Data represent the mean±SD from three experiments. * p≤0.05; ** p≤0.01 and *** p≤0.001; and

FIG. 2D shows the expression of antiviral genes, indicating that the addition of EVs to the infected cells enhances the expression of antiviral genes in both AECs and HMVECs. Data represent the mean±SD from three experiments. * p≤0.05; ** p≤0.01 and *** p≤0.001.

### DETAILED DESCRIPTION

Severe H5N1 influenza virus infection in humans cause significant mortality that is mainly attributed to acute lung injury, for which few current therapeutic options are available.

Accordingly, the present invention utilizes primary human alveolar epithelial cells and human microvascular endothelial cells to establish a highly physiologically relevant preclinical model for the study of respiratory pathogens in an in vitro setting.

In particular, the present invention provides an in vitro co-culture alveolus-capillary model system for studying acute lung injury induced by respiratory virus infections. The system includes a transwell system with primary human alveolar epithelial cells seeded onto an apical chamber surface of the transwell system and human lung microvascular endothelial cells seeded onto a basolateral surface of the transwell system, and an apparatus for measuring alveolar fluid clearance and alveolar protein permeability in infected cells. The in vitro co-culture alveolus-capillary model system mimics an alveolo-capillary barrier. The primary human alveolar epithelial cells are cultured on a collagen-coated transwell insert to promote tight junction formation.

This model surpasses existing systems by utilizing primary human AECs and HMVECs cultured on a collagen-coated transwell system, thereby more accurately replicating the alveolus-capillary barrier of human lungs. The co-culture model is good for patient-specific therapy as the primary human lung cells are acquired from a specific donor, which allows tailored treatment of respiratory diseases based on the fine-tuning of phenotypic drug screening in the lung model. This can lead to direct translation into the clinical setting, therefore maximizing the chance of successful treatment.

The co-culture alveolar-capillary model formed by human AECs and HMVECs offers several significant advantages. First, it provides enhanced physiological relevance by reconstructing the barrier between alveoli and capillaries, capturing the epithelial-endothelial interactions that are critical for studying lung diseases. This model yields more accurate results than traditional monoculture systems, particularly in studies of lung injury caused by infections.

Unlike traditional models that use immortalized cell lines (such as A549), this system employs primary cells isolated from human tissues, resulting in a more accurate physiological representation. The use of primary cells also offers the potential for patient-specific therapy, as cells can be obtained from individual patients, allowing for personalized treatment assessments.

In one embodiment, the transwell system has a pore size of approximately 0.4 μm. This pore size is used for assessing the rate of AFC and APP by measuring the migration of FITC-dextran (70 kDa) from the apical chamber into the basolateral chamber of transwells.

The transwell system in this invention enables a dual-layer co-culture in which the AECs are seeded on the apical chamber surface, while the HMVECs are cultured on the basolateral surface. This mimics the natural structure and functions of the human alveolo-capillary barrier. The primary human alveolar epithelial cells are seeded at a cell density of 5×10⁴ to 2×10⁵ cells per well, and the human lung microvascular endothelial cells are seeded at a cell density of 5×10⁴ to 2×10⁵ cells per well.

Preferably, the primary human alveolar epithelial cells are seeded at a cell density of 1×10⁵ cells per well, and the human lung microvascular endothelial cells are seeded at a cell density of 1×10⁵ cells per well.

The cell density required for HMVECs in OOAC is twice that described in this invention. It appears that the transwell system of the present invention remains the mainstream of cellular co-culture as it is easily accessible without the need for specialized equipment, while maintaining the physiological relevance required for accurate data acquisition.

One of the significant advantages of this invention is its ability to measure the integrity of the epithelial-endothelial barrier by monitoring TEER. This is an important numerical standard, as the TEER value within this range indicates that the cell layer has formed functional, intact, and stable tight junctions, exhibiting good barrier function.

In one embodiment, the TEER are consistently maintained above 800 Ω/cm², confirming strong tight junction formation. When the TEER value exceeds 800 Ω/cm², the system is capable of adequately simulating the alveolar-capillary interface of the human body, indicating that the tight junction integrity between epithelial and endothelial cells is sufficient to resist external stimuli or pathogen invasion.

Moreover, the present invention also provides a method for in vitro studying of acute lung injury induced by respiratory virus infections, including: isolating primary human alveolar epithelial cells and human microvascular endothelial cells; seeding the primary human alveolar epithelial cells on an apical chamber surface of a transwell membrane and seeding the human microvascular endothelial cells on a basolateral surface of the transwell system to form an in vitro co-culture alveolus-capillary model system; infecting the in vitro co-culture alveolus-capillary model system with one or more respiratory virus; and assessing effect of viral infection on at least one physiological parameter comprising AFC and APP.

In one embodiment, the one or more respiratory virus may include coronaviruses, respiratory syncytial viruses, adenoviruses, influenza viruses, parainfluenza viruses, rhinoviruses, enteroviruses, and herpes simplex viruses.

Preferably, the one or more respiratory virus is influenza virus A (H5N1).

The influenza A (H5N1) viruses significantly impair AFC and APP in the co-culture, inducing high levels of influenza viral gene activity that disrupt tight junctions between cells. This also hinders the expression of antiviral genes required to mitigate viral damage to the barrier.

EVs derived from human mesenchymal stromal cells are utilized as a therapeutic agent. Upon infection, the administration of EVs has been shown to significantly restore AFC and reduce APP, mitigating the damage caused by viral replication.

In addition, the administration of EVs to the A (H5N1) virus-infected epithelium effectively reduces viral replication by enhancing the expression of antiviral genes (e.g., ISG15 and MX-1) in both the epithelium and endothelium. This, in turn, improves AFC and APP, bringing them closer to normal levels.

The experimental results indicate the therapeutic potential of EVs and highlight the usability of the in vitro co-culture system for examining the epithelial and endothelial interactions in the pathogenesis of acute lung injury in a patient-specific scenario. In addition, it can be used to improve techniques of lung drug delivery that also requires a functional barrier.

### EXAMPLE

### Example 1

### Materials and Methods

Influenza virus A/HK/483/97 (H5N1) is used for infection. All influenza viruses are passaged in Madin-Darby Canine Kidney (MDCK) cells. Viral titers are determined by median tissue culture infectious dose (TCID₅₀). All experiments are performed inside a biosafety level-3 facility.

### Isolation of Human AECs

Human AECs are isolated from resected, non-malignant human lung tissue. Briefly, the visible bronchi are removed, and the lung tissue is minced into pieces of less than 0.5 mm in size. The minced tissue is washed three times with Hank's balanced salt solution (HBSS) (Gibco) and 0.7 mM sodium bicarbonate (Gibco) at pH 7.4 to remove most of the macrophages and blood cells. The tissue is digested using a combination of 0.5% trypsin (Gibco) and 4 U/mL elastase (Worthington Biochemical Corp.) for 40 minutes at 37° C. in a shaking water bath.

The digestion is stopped by adding DMEM/F12 medium (Gibco) with 40% FBS and 350 U/mL DNase I (Sigma). Cell clumps are dispersed by repeatedly pipetting the cell suspension for 10 minutes. A disposable cell strainer (gauze size 50 μm) (BD Bioscience) is used to remove large undigested tissue fragments. The single-cell suspension is centrifuged, and the pellet is resuspended in a 1:1 mixture of DMEM/F12 medium and small airway basal medium (Lonza) supplemented with 0.5 ng/ml epidermal growth factor, 500 ng/ml epinephrine, 10 μg/mL transferrin, 5 μg/mL insulin, 0.1 ng/ml retinoic acid, 6.5 ng/ml triiodothyronine, 0.5 μg/mL hydrocortisone, 30 μg/mL bovine pituitary extract, 0.5 mg/mL BSA, 5% FBS and 350 U/mL DNase I. The cell suspension is plated in tissue culture flasks and incubated in a 37° C. water-jacketed incubator at 5% CO₂ for 90 minutes. The non-adherent cells are layered on a discontinuous cold Percoll density gradient (density 1.089 and 1.040 g/mL) and centrifuged at 25×g for 20 minutes. The cell layer at the interface of the two gradients is collected and washed four times with HBSS to remove the Percoll fluid. The cell suspension is incubated with magnetic beads coated with anti-CD14 antibodies (MACS CD14MicroBeads) at room temperature for 20 minutes.

After removal of the beads by using a magnet, cell viability is assessed by trypan blue dye exclusion. The alveolar epithelial cell suspension is resuspended in SAGM (Lonza) supplemented with 1% FBS, 100 U/mL penicillin and 100 μg/mL streptomycin and plated at a cell density of 3×10⁵ per cm². The cells are maintained in a humidified atmosphere (5% CO₂, 37° C.), and growth medium is changed daily, starting 60 hours after plating the cells. When the cell layer approached 75% confluence, the alveolar epithelial cells are dissociated with trypsin, detached into HBSS, and then seeded as described below in the transwell culture inserts.

### Isolation of HMVECs

The HMVECs are isolated from human non-tumor lung tissue. Briefly, lung tissues are minced and digested with 1% collagenase type II (Thermo Fisher scientific), dispase (Thermo Fisher scientific) and DNase I (Sigma) for 1 hour at 37° C. with shaking. The digestion is quenched with DMEM with 10% FBS. The digestion mixture is strained through a 70-μm cell strainer (BD Falcon) and then the HMVECs are separated from other cell types (e.g. red blood cells, granulocytes and other cell debris) using Ficoll-Paque Plus (GE Healthcare). After centrifugation at 400×g for 20 minutes, all the interphase cells containing HMVECs are retrieved. The cells are incubated with biotinylated anti-CD31 antibody (Abcam) tagged streptavidin-tagged M-280 Dynabeads (Thermo Fisher scientific) for 30 minutes at 4° C. with gentle agitation. After incubation, the bead-bound-cells are washed with 1 ml MACS buffer 5times using a DynaMag5 magnet (Thermo Fisher scientific). The bead-bound CD31-positive cells are resuspended in endothelial cell growth medium-2, EGM-2 (Lonza), and plated on a 0.2% gelatin-coated tissue culture flask for culture. The cells are maintained in a humidified atmosphere (5% CO₂, 37° C.), and growth medium is changed every two days. When the cell layer approached 75% confluence, the endothelial cells are dissociated with trypsin, detached into HBSS, and then seeded as described below in the transwell culture inserts.

### Statistical Analysis

All in vitro experiments are conducted independently in triplicates. Groups are compared by using an unpaired, two-tailed t test. Differences are considered significant if p≤0.05.

### Example 2

### Generation of In Vitro Co-Culture Alveolus-Capillary Model

To study the effect of influenza viruses on alveolar fluid clearance and protein permeability in human AECs and HMVECs, this example modifies a physiologically relevant 24-transwell in vitro acute lung injury model to create a co-culture model suitable for use in a BSL-3 laboratory.

Rat-tail collagen is first coated onto the apical chamber surface of the 24-well Costar Transwell inserts with a 0.4-μm pore size (Corning) and allowed to set. Then, 1×10⁵ HMVECs are seeded onto the basolateral surface of the transwell membrane and allowed to attach for 2 hours. Following this, 1×10⁵ AECs are seeded onto the apical chamber surface of the membrane and permitted to attach overnight. Plated cells are maintained in a humidified atmosphere (5% CO₂, 37° C.), and the culture media is refreshed every 48 hours. Transepithelial resistance is maintained at ≥800 Ω/cm², which indicates good tight junction integrity between cells.

Cytomix, a mixture of the cytokines IL-1β, TNF-α, and IFN-γ (R&D Systems), is used at 50 ng/ml as a positive control for lung injury.

### Example 3

### Measurement of AFC and APP

Net alveolar fluid transport from the apical chamber of the transwell system to the basolateral chamber of the transwell system at 24 hours post infection is measured.

The AECs are inoculated with the respective influenza A viruses at a multiplicity of infection (MOI) of 0.1 for 1 hour. Then, 200 μl of FITC-labeled dextran (Sigma) with a size of 70 kDa is added to the cells (final concentration, 500 ng/μl). After 5 minutes, a sample of 100 μl is collected from the apical chamber for initial FITC measurement, after which it is transferred back into the apical chamber for overnight incubation. After 24 hours of dextran incubation, 100 μl is collected from the apical chamber, while 100 μl is collected from the basolateral chamber for the final FITC measurement. The fluorescence of each sample is measured by a modulus fluorometer (FLUOstar OPTIMA, BMG Labtech) at excitation wavelength of 485 nm and emission wavelength of 520 nm. A standard curve of known FITC concentration is constructed for the calculation of FITC-dextran present in the transwell chambers at different time points. The net alveolar fluid transport across the epithelial monolayer is measured as:

[1−(FITC concentration in the initial apical sample/FITC concentration in the final apical sample)]/200 μl/0.33 cm²/24 hours

The final basolateral reading is collected after 24 hours of dextran incubation to measure protein permeability, based on the unidirectional flux of fluorescence-labeled dextran from the apical chamber to the basolateral chamber of the transwell system.

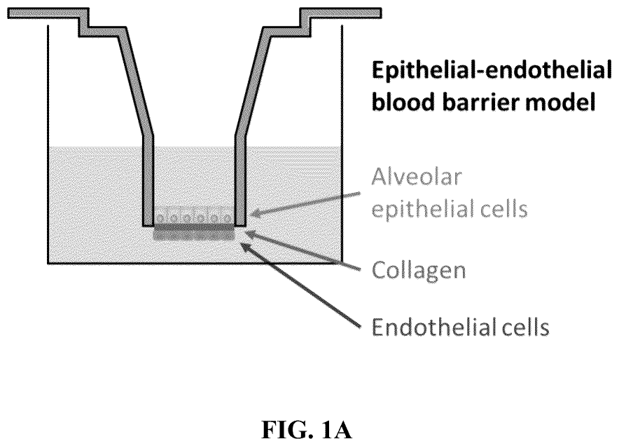

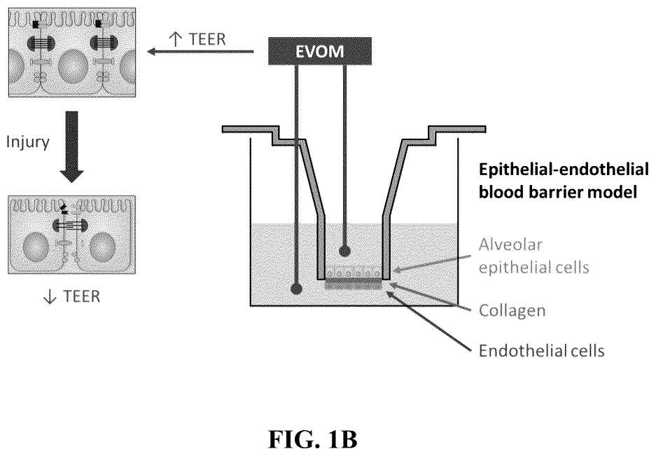

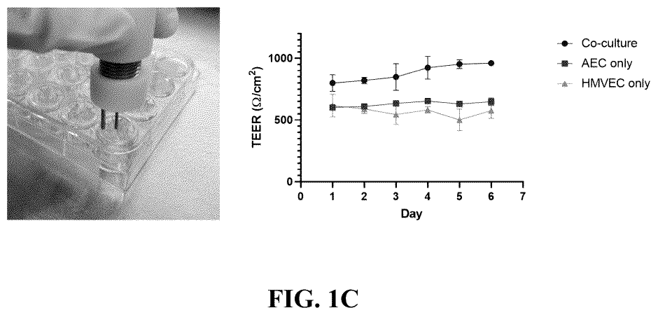

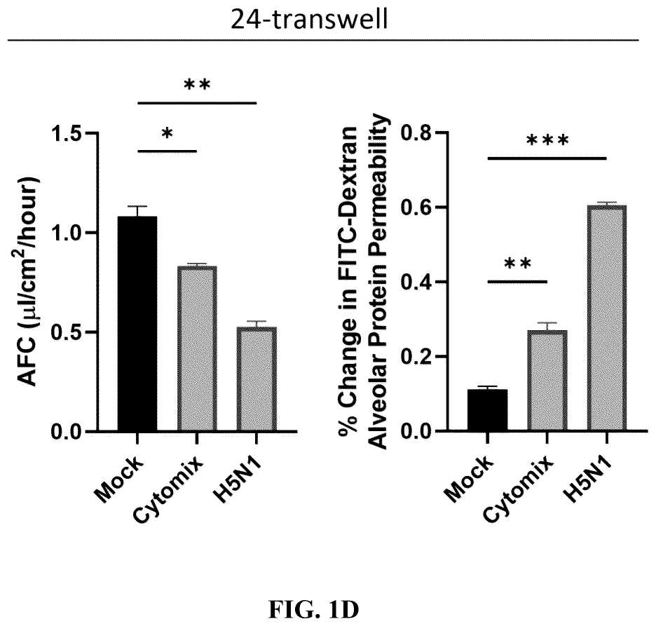

FIGS. 1A-1D depict the development of an in vitro alveolus-capillary model system. Monolayers of primary human AECs and HMVECs are grown on the apical (rat-tail collagen-coated) and basolateral chamber surfaces of the transwell membrane, respectively. This setup serves as a physiologically relevant model of the human lung alveolus-capillary interface, enabling the study of fluid transport across the epithelial-endothelial barrier in vitro (FIG. 1A).

The integrity of tight junctions between cells is monitored daily using EVOM machine. Referring to FIG. 1B, as tight junctions form, the TEER increases, while damage to the monolayer results in a decrease in TEER. The TEER steadily increases over a 6-day period before experiments are conducted on the established in vitro model system. Referring to FIG. 1C, the TEER values from the co-culture of both AECs and HMVECs are consistently higher than those from either cell type grown alone.

FIG. 1D shows that influenza A (H5N1) significantly reduces AFC and increases APP at 24 hours post-infection compared to mock group. A mixture of proinflammatory cytokines [Cytomix (IL-1β, TNF-α, and IFN-γ)] serves as positive control for monolayer damage.

### Example 4

### Nano-Sized EVs Prevent A (H5N1) Virus Impairment of AFC and APP via Suppression of Viral Replication in Both AECs and HMVECs

In this example, the AECs in the apical chamber are infected with A (H5N1) influenza virus at a multiplicity of infection (MOI) of 0.1 to monitor AFC and APP. Additionally, an MOI of 2 is used to study the expression of viral and antiviral genes in both AECs and HMVECs following infection.

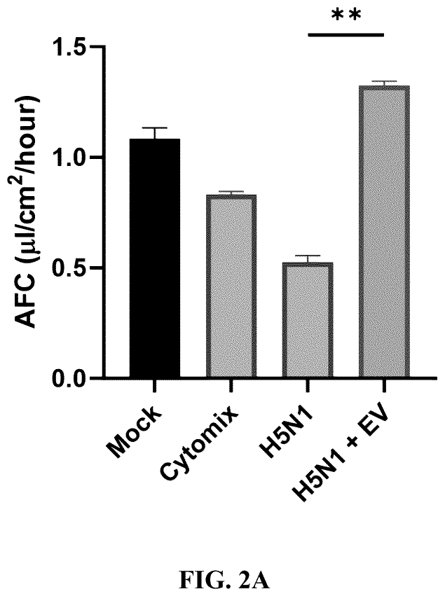

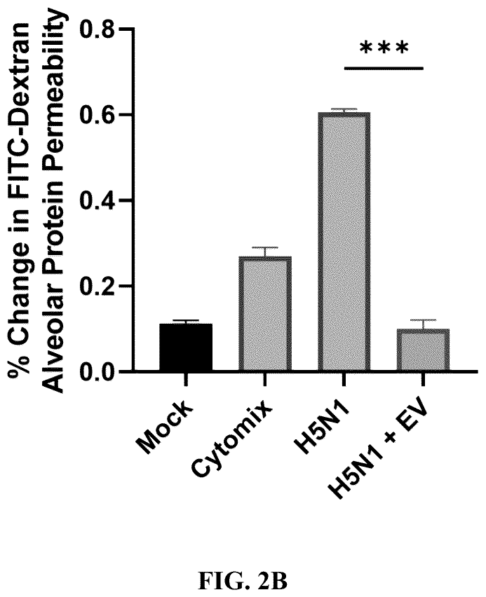

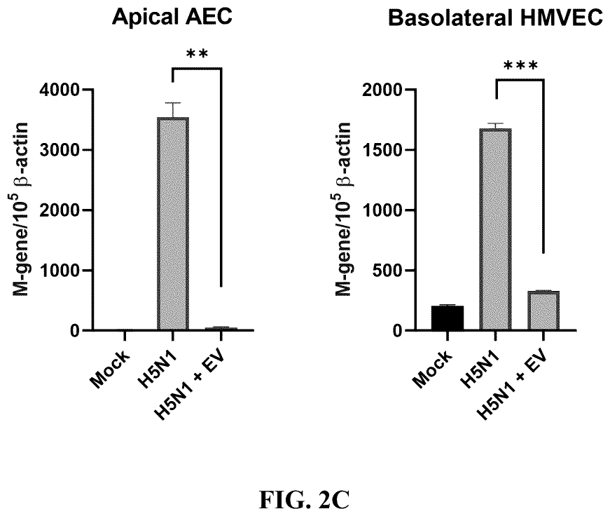

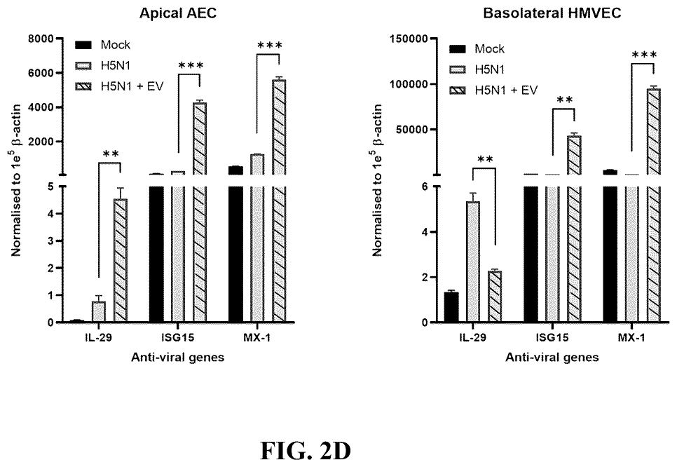

FIGS. 2A-2D depict the antiviral activity of nano-sized therapeutic EVs, which help prevent A (H5N1) virus-induced impairment of alveolar epithelial-endothelial AFC and APP. First, the AEC in the transwells are infected with A (H5N1) influenza viruses. The results demonstrate that the addition of EVs to A (H5N1)-infected cells restores AFC (FIG. 2A) and reduce APP (FIG. 2B) compared to untreated infected cells.

As shown in FIG. 2C, EV treatment suppresses the elevated expression of the viral M-gene following A (H5N1) infection in the AECs. While the AECs are the first to be exposed to the influenza virus, the underlying HMVECs are also susceptible to A (H5N1) infection. This leads to similar patterns of viral gene expression in HMVECs compared to the epithelium, albeit at lower levels. This suppression correlates with an increase in the expression of the antiviral genes ISG15 and MX-1 induced by EV treatment (FIG. 2D). An interesting observation is the differential expression of IL-29 between epithelial and endothelial cells, indicating that when the influenza virus infiltrates beyond the epithelium, the endothelium acts as a secondary barrier of defence to prevent the virus from entering the bloodstream.

In summary, the present invention establishes a physiologically relevant in vitro alveolus-capillary lung injury model to investigate the pathogenesis of severe human influenza through epithelial-endothelial cell interactions. By using this model, it demonstrates that highly pathogenic A (H5N1) viruses significantly impair AFC and APP in the epithelial-endothelial barrier by boosting virus replication that increased virus-induced damage to the barrier integrity. This pathology is subdued by the addition of EVs to virus-infected AEC in vitro, indicating their therapeutic potential for treating diseases arising from A (H5N1) infection and to avert from the development of more serious complications.

The donor-specific nature of the primary cells used also provides a platform for investigating patient-specific influenza virus-induced pathogenesis and tailored therapy that can be readily translated into a clinical setting.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### Definition

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

“Transepithelial Electrical Resistance (TEER)” is a quantitative measurement of the electrical resistance across a monolayer of cells, typically used to assess the integrity and permeability of tight junctions in epithelial or endothelial cell cultures. TEER provides a direct indicator of the strength of the barrier function formed by the cells. Higher TEER values indicate stronger tight junctions and lower permeability, reflecting a more intact and functional epithelial or endothelial barrier, while lower TEER values suggest weakened junctions and increased permeability. In in vitro models, TEER is commonly used to monitor the health and integrity of cell monolayers over time.

“Extracellular vesicles (EVs)” refers to nano-sized particles secreted by cells that facilitate intercellular communication and can have therapeutic effects by transferring biomolecules between cells.

“Alveolar Fluid Clearance (AFC)” refers to the process by which fluid is actively removed from the alveolar space (the small air sacs in the lungs) into the lung interstitium, and eventually absorbed into the bloodstream or lymphatic system. Proper AFC function is crucial for preventing pulmonary edema (excess fluid in the lungs).

“Alveolar Protein Permeability (APP)” describes the ability of proteins, particularly plasma proteins, to pass through the alveolar-capillary barrier (composed of the alveolar epithelium and the capillary endothelium). Increased APP can indicate damage or increased permeability of this barrier, leading to the leakage of proteins and fluids into the alveolar space, which can impair lung function and contribute to conditions like acute respiratory distress syndrome (ARDS).

Other definitions for selected terms used herein may be found within the detailed description of the present invention and apply throughout. Unless otherwise defined, all other technical terms used herein have the same meaning as commonly understood to one of ordinary skill in the art to which the present invention belongs.

What is claimed is:

1. An in vitro co-culture alveolus-capillary model system for studying acute lung injury induced by respiratory virus infections, wherein the in vitro co-culture alveolus-capillary model system comprises:
a transwell system with primary human alveolar epithelial cells seeded onto an apical surface of the transwell system and human lung microvascular endothelial cells seeded onto a basolateral surface of the transwell system; and
an apparatus for measuring alveolar fluid clearance and alveolar protein permeability in infected cells, wherein the in vitro co-culture alveolus-capillary model system mimics an alveolo-capillary barrier.

2. The in vitro co-culture alveolus-capillary model system of claim 1, wherein the primary human alveolar epithelial cells are cultured on a collagen-coated transwell insert to promote tight junction formation.

3. The in vitro co-culture alveolus-capillary model system of claim 1, wherein the primary human alveolar epithelial cells are isolated from a non-malignant lung tissue.

4. The in vitro co-culture alveolus-capillary model system of claim 1, wherein the primary human alveolar epithelial cells are seeded at a cell density of 5×10⁴ to 2×10⁵ cells per well.

5. The in vitro co-culture alveolus-capillary model system of claim 1, wherein the human lung microvascular endothelial cells are seeded at a cell density of 5×10⁴ to 2×10⁵ cells per well.

6. The in vitro co-culture alveolus-capillary model system of claim 1, wherein the transwell system has a pore size of 0.4 82 m.

7. A method for in vitro studying acute lung injury induced by respiratory virus infections, comprising:
isolating primary human alveolar epithelial cells and human microvascular endothelial cells;
seeding the primary human alveolar epithelial cells on an apical surface of a transwell membrane and seeding the human microvascular endothelial cells on a basolateral surface of the transwell system to form an in vitro co-culture alveolus-capillary model system;
infecting the in vitro co-culture alveolus-capillary model system with one or more respiratory virus;
assessing effect of viral infection on at least one physiological parameter comprising alveolar fluid clearance (AFC) and alveolar protein permeability (APP).

8. The method of claim 7, wherein the primary human alveolar epithelial cells and the human microvascular endothelial cells are isolated using enzymatic digestion followed by density gradient centrifugation and magnetic bead separation.

9. The method of claim 7, wherein the primary human alveolar epithelial cells are cultured on a collagen-coated transwell insert to promote tight junction formation.

10. The method of claim 7, wherein the primary human alveolar epithelial cells are isolated from a non-malignant lung tissue.

11. The method of claim 7, wherein the primary human alveolar epithelial cells are seeded at a cell density of 5×10⁴ to 2×10⁵ cells per well.

12. The method of claim 7, wherein the human lung microvascular endothelial cells are seeded at a cell density of 5×10⁴ to 2×10⁵ cells per well.

13. The method of claim 7, wherein the transwell system has a pore size of 0.4 μm.

14. The method of claim 7, wherein the one or more respiratory virus are selected from the group consisting of coronaviruses, respiratory syncytial viruses, adenoviruses, influenza viruses, parainfluenza viruses, rhinoviruses, enteroviruses, and herpes simplex viruses.

15. The method of claim 14, wherein the in vitro co-culture alveolus-capillary model system is infected by influenza A virus subtype H5N1.

16. The method of claim 7, further comprising adding a therapeutic agent after the viral infection to inhibit viral replication within both the human alveolar epithelial cells and the human lung microvascular endothelial cells.

17. The method of claim 16, wherein the therapeutic agent comprises extracellular vesicles in a concentration of 1×10⁷ to 1×10⁹ EV particles per well.

18. The method of claim 17, wherein the extracellular vesicles are derived from human mesenchymal stromal cells.

19. The method of claim 7, further comprising measuring transepithelial electrical resistance (TEER) as an indicator to assess the integrity of the epithelial-endothelial barrier over time, and the TEER is maintained at ≥800 Ω/cm².

20. The method of claim 7, wherein step of assessing effect of viral infection on at least one physiological parameter further comprises measuring viral gene expression and anti-viral gene expression in both the primary human alveolar epithelial cells and the human microvascular endothelial cells.
